(19) United States
(12) Patent Application Publication
(10) Pub. No.: US 2021/0186889 A1
(43) Pub. Date: Jun. 24, 2021

(54) INTESTINAL-RELEASE FORMULATION OF A DIGESTIVE ENZYME, METHOD OF PRODUCTION AND GALENIC PREPARATION

(71) Applicant: ENEAPHARM, LABEGE (FR)

(72) Inventors: Karim IOUALALEN, SAINT ORENS DE GAMEVILLE (FR); Rose-Anne RAYNAL, SAINT ORENS DE GAMEVILLE (FR)

(21) Appl. No.: 17/040,360

(22) PCT Filed: Mar. 14, 2019
(86) PCT No.: PCT/FR2019/050557

§ 371 Date: Jan. 20, 2021

(30) Foreign Application Priority Data
Mar. 23, 2018 (FR) 18 70330

(51) Int. Cl. A61K 9/51; A61K 38/54

(52) U.S. Cl. CPC A61K 9/5123; A61K 9/5192; A61K 38/54

(57) ABSTRACT

The invention relates to a formulation of at least one digestive enzyme, the formulation comprising solid lipid particles, the solid lipid particles being:
between 50 μm and 1200 μm in size,
strictly hydrophobic,
free of water, of organic solvent, of surfactant compound and of polymer, and comprising:
at least one digestive enzyme,
a strictly hydrophobic, non-hygroscopic waxy matrix, in which said at least one digestive enzyme:
has homogeneous distribution in each solid lipid particle of the formulation,
is distributed without a distribution gradient towards the interior of the waxy matrix, and
represents between 0.1% and 90% of the mass of the formulation, the formulation having a melting point of between 20° C. and 65° C.

The invention relates to a formulation for the intestinal release of at least one digestive enzyme. The invention is directed towards such a formulation, named gastro-protective formulation, for protecting at least one digestive enzyme during its transit through the stomach and for the in vivo restoration of intestinal digestive function.

FR2913884 discloses a non-ionizable hydrophobic galenical system.

The present invention is directed towards such a gastro-protective formulation, in the form of solid, strictly hydrophobic, non-hygroscopic, non-injectable lipid particles, not containing any water, not containing any surfactant, not containing any emulsifier, not containing any trace of solvent, not containing any trace of shear-thinning hydrophilic polymer, and containing at least one enzyme for establishing exocrine pancreatic enzymatic activity in the intestine in compensation for deficient exocrine pancreatic activity. Such a formulation and the galenical forms intended for the oral administration of such a formulation are intended for treating exocrine pancreatic insufficiency.

The treatment of exocrine pancreatic insufficiency (EPI) is based on the oral administration of pancreatic enzymes (PERT), or pancreatin, for overcoming the absence of production of exocrine digestive enzymes by the pancreas.

The medicinal substance pancrelipase is mainly a combination of three classes of enzymes: lipase, protease and amylase, and also the various cofactors and coenzymes thereof These enzymes are naturally produced in the pancreas and are necessary for the digestion of fats, proteins and carbohydrates. Medicinal pancrelipase is typically prepared from porcine pancreatic glands. Other sources of pancrelipase may be used, for example those described in U.S. Pat. No. 6 051 220, US 2004/0057944 and WO 2006/044529. The enzymes catalyse the hydrolysis of fats to glycerol and fatty acids, starch to dextrin and sugars, and proteins to amino acids and derived substances. The native pancreatic enzymes are extremely acid-sensitive, and so the oral supply of pancreatic enzymes to compensate for exocrine pancreatic enzymes poses a problem. Such orally-supplied enzymes are degraded during their passage through the stomach. Lipases are the most sensitive to acid-mediated inactivation. To enable the oral administration of digestive enzymes that are capable of reaching the intestinal tract, the industry has developed various formulation processes, by polymeric coating as described in U.S. Pat. Nos. 8,221,747 and 8,246,950.

Coating techniques, or “polymeric coating”, consist in producing an outer layer of insulating compounds, of polymers or of mixtures, around the active principle to insulate it from the external medium, as described in the patents WO 00/30617 and WO 02/092106. Many natural or synthetic polymeric compounds have been used to make this outer layer. Cellulose derivatives such as hydroxypropylmethylcellulose (HPMC), ethylcellulose, CMCs, HPMC phthalate and mixtures of these products, but also acrylate polymers and derivatives thereof, are mainly distinguished. This technique has given interesting results for the preparation of sustained-release forms and for gastro-protection. Such a formulation in water remains unstable over time, which makes it incompatible with the preparation of aqueous forms such as syrups and suspensions.

However, these formulations used for pancrelipases have a certain number of drawbacks, as indicated in the document World I Gastroenterol. 2014 Sep. 7; 20(33): 11467-1148. The therapeutic response of products using these formulations is low. The mean particle size is greater than 1.5 mm, bringing about segregation with the foods in the stomach and causing insufficient mixing in the intestinal tract. Finally, the release of the enzymes into the intestine is insufficient and too slow.

There is thus a need for an enteric formulation with rapid and complete release of pancrelipase and/or of pancreatic enzyme, the particle size of which is less than 1 5 mm and which is compatible with facilitated intake by the patient, in particular by paediatric patients, to ensure better compliance with the treatment.

The invention is directed towards overcoming these drawbacks.

The invention is also directed towards proposing a novel formulation for the administration of digestive enzymes, enabling:
protection of the digestive enzymes against the conditions encountered in the stomach (gastro-protection),
masking of the taste, enabling the preparation of extemporaneous taste-masked aqueous forms,
rapid release into the intestinal medium,
masking of the mucosal irritability effects.

The invention relates to a formulation, notably a formulation for the intestinal release, of at least one digestive enzyme, the formulation comprising solid lipid particles,
the solid lipid particles being:
between 50 μm and 1200 μm (micrometres) in size,
strictly hydrophobic,
free of water, of organic solvent, of surfactant compound and of polymer, in particular of surface polymer, and comprising:
at least one digestive enzyme, notably at least one digestive enzyme chosen from the group formed from a pancrelipase, pancreatic enzymes and analogues thereof, and
a strictly hydrophobic, non-hygroscopic waxy matrix, in which said at least one digestive enzyme:
has homogeneous distribution in each solid lipid particle of the formulation,
is distributed without a distribution gradient towards the interior of the waxy matrix, and
represents between 0.1% and 90% of the mass of the formulation, the formulation having a melting point of between 20° C. and 65° C.

The inventors have discovered, entirely unexpectedly, that strictly hydrophobic solid lipid particles may be used stably for the preparation of a formulation with rapid release of pancreatic digestive enzyme(s) or of pancreatic enzyme analogue(s), which is enteric, i.e. which enables protection of the digestive enzymes, and without the solid lipid particulate structure being substantially modified during the gastric phase.

The formulation according to the invention is characterized in that it has the capacity to protect, during the gastric phase, digestive enzymes delivered orally while at the same time allowing rapid and more complete release in the intestinal digestive tract. Despite the presence of lipase, the system remains stable and functional while at the same time masking the taste and the odour.

The invention relates to a formulation in the form of solid, strictly hydrophobic lipid particles, containing one or more enzymes for re-establishing exocrine pancreatic enzymatic function in the intestine.

The formulation in the form of solid lipid particles according to the invention is characterized in that:
it comprises a strictly hydrophobic and non-hygroscopic waxy matrix,
it is strictly hydrophobic and gastro-protected,
it is free of any trace of aqueous or organic solvent,
it is free of surfactant or amphiphilic or detergent compounds,
it is free of polymer(s), in particular of surface polymer(s), and of residue(s) of such polymeric agent(s). The solid lipid particles are produced without the need to use such polymers,
it is characterized by homogeneous distribution of said at least one digestive enzyme in the waxy matrix without a radial gradient,
it is biocompatible and dispersible in intestinal medium,
it cannot be administered parenterally,
it contains said at least one digestive enzyme, notably a pancrelipase or one or more pancreatic enzymes or pancreatic extracts, or similar enzymes, alone or as mixtures, and optionally cofactors and coenzymes, and
it makes it possible, besides affording gastro-protection, to improve the stability of said at least one digestive enzyme and to accelerate the intestinal release while ensuring masking of the taste and of the odour.

Advantageously, formulation consists solely of solid lipid particles, the solid lipid particles being formed solely from at least one digestive enzyme and from a strictly hydrophobic, non-hygroscopic waxy matrix.

Advantageously, the solid lipid particles have a size of between 150 μm and 800 μm and notably between 250 μm and 550 μm. They are strictly hydrophobic, containing one or more enzymes for re-establishing the digestive function in the intestine.

Advantageously, the formulation has a melting point of between 20° C. and 65° C., notably a melting point of between 20° C. and 55° C., preferably between 30° C. and 50° C., even more preferentially between 32° C. and 48° C.

Advantageously, the waxy matrix has a melting point of between 20° C. and 65° C., notably a melting point of between 20° C. and 55° C., preferably between 30° C. and 50° C., even more preferentially between 32° C. and 48° C. In the text hereinbelow, the terms “lipid particle” and “lipid granule” will be understood as having the same meaning

A waxy matrix having a suitable composition, which is compatible in terms of toxicity, biocompatibility, absence of immunogenicity and biodegradability, with oral absorption is chosen. The components of the waxy matrix will be chosen from components already used for oral administration, such as those defined in the GRAS list published by the Food and Drug Administration, and such that the solid lipid particles formed have properties of incorporating, taste-masking, stabilizing and releasing said at least one digestive enzyme.

At least one digestive enzyme is pancrelipase, also known as pancreatin. Throughout the text, the terms “pancrelipase” and “pancreatin” denote a mixture of several types of enzymes, the amylase, lipase and protease enzymes. A pancrelipase or pancreatin may be obtained by extraction from the pancreas. It may be produced artificially, or obtained from sources other than the pancreas, for example from microbes, plants or other animal tissues. When it originates from a pancreatic extract, pancrelipase is associated with cofactors. Pancrelipase is commercially available, for example from Nordmark Arzneimittel or Scientific Protein Laboratories.

Advantageously and according to the invention, said at least one formulated digestive enzyme is stabilized.

In one embodiment of a formulation according to the present invention, said at least one digestive enzyme comprises a lipase. The term “lipase” denotes an enzyme which catalyses the hydrolysis of lipids into glycerol and simple fatty acids.

Examples of lipases that are suitable for performing the present invention comprise an animal lipase, for example a lipase of porcine or bovine origin, a bacterial lipase, for example a lipase from Pseudomonas bacteria (Pseudomonas lipase), a fungal lipase, a plant lipase, a recombinant lipase, a chemically modified lipase, or a mixture thereof.

In one embodiment of a formulation according to the present invention, at least one formulated digestive enzyme comprises an amylase. The term “amylase” denotes glycoside hydrolase enzymes which decompose starch, for example α-amylases, β-amylases, γ-amylases, α-glucosidase acids and salivary amylases.

The amylases that may be used in a formulation according to the present invention are chosen from the group formed from animal amylases, bacterial amylases, fungal amylases, for example an amylase from Aspergillus fungi, an amylase from Aspergillus oryzae, plant amylases, chemically modified amylases, or mixtures thereof.

In one embodiment of a formulation according to the present invention, at least one digestive enzyme is a protease. The term “protease” denotes enzymes, for example proteinases, peptidases or proteolytic enzymes, which break the peptide bonds between the amino acids of proteins. Proteases are generally identified by their catalytic type, for example aspartic acid peptidases, cysteine(thiol)-peptidases, serine-peptidases, threonine-peptidases, alkaline or semi-alkaline proteases, neutral proteases and novel peptidases.

As examples of proteases that may be used in a formulation according to the present invention, mention may be made of serine-proteases, threonine-proteases, cysteine-proteases, aspartic acid proteases, without limitation of the type of proteases. A formulation according to the present invention may comprise at least one animal protease, at least one bacterial protease, at least one fungal protease, at least one plant protease, at least one recombinant protease, at least one chemically modified protease, alone or as mixtures.

In the description, the term “recombinant enzymes” denotes enzymes produced by DNA recombination by a suitable host cell, chosen from any one from among host cells of bacteria, yeasts, fungi, plants or animals, or recombinant enzymes comprising an amino acid sequence that is homologous or substantially identical to a natural sequence of enzymes or comprising a nucleic acid that is homologous or substantially identical to a nucleic acid coding for the natural enzyme.

The formulations according to the present invention may comprise one or more lipases, one or more amylases, one or more proteases, mixtures comprising one or two or each of the various types of enzymes.

In one embodiment, said at least one digestive enzyme is a porcine or bovine pancreatic extract comprising various lipases, for example a lipase, a colipase, a phospholipase A2, a cholesterol esterase, proteases, for example a trypsin, a chymotrypsin, a carboxypeptidase A and/or B, an elastase, a kininogenase, a trypsin inhibitor, amylases, and optionally nucleases—ribonuclease or deoxyribonuclease. In one embodiment, said at least one digestive enzyme is a mixture substantially similar to human pancreatic fluid. In yet another embodiment, at least one digestive enzyme is pancrelipase USP.

The lipase activities in the compositions and the forms of the present invention may be from about 100 to 100 000 IU, and in a particular embodiment from 500 to 25 000 IU per administrable unit dose.

The amylase activities in the compositions according to the invention are between 100 and 320 000 IU per therapeutic unit or unit dose, and preferentially between 1500 and 75 000 IU.

The protease activities in the compositions or the forms of the present invention may be between 100 and 180 000 IU, and preferentially between 1000 and 80 000 IU per unit dose.

In one embodiment, the lipase activity is between about 400 and 500 IU, the amylase activity is between about 1750 and 2750 IU and the protease activity is between about 1200 and 2000 IU.

In one embodiment, the lipase activity is between about 4000 and 5000 IU, the amylase activity is between about 17 500 and 27 500 IU and the protease activity is between about 12 000 and 20 000 IU.

In another embodiment, the lipase activity is between about 10 000 and 12 500 IU, the amylase activity is between about 22 000 and 90 000 IU and the protease activity is between about 17 000 and 75 000 IU.

In yet another embodiment, the lipase activity is between about 12 500 and 15 500 IU, the amylase activity is between about 25 000 and 90 000 IU and the protease activity is between about 26 000 and 100 000 IU per unit dose.

In yet another embodiment, the lipase activity is between about 17 000 and 25 000 IU, the amylase activity is between about 40 000 and 185 000 IU and the protease activity is between about 30 000 and 125 000 IU per unit dose.

Thus, one subject of the invention is a formulation of digestive enzymes in a waxy matrix in the form of solid lipid particles, for re-establishing the exocrine pancreatic enzymatic function in the intestine.

The waxy matrix of the solid lipid particles of a formulation according to the invention consists of a hydrophobic compound or of a mixture of hydrophobic compounds which is (are) insoluble in water, solid at room temperature and totally free of surfactant compounds, solvent residues and water. Thus, any hydrolysis or oxidation reaction is avoided. By way of example, mention may be made of hydrophobic waxes or mixtures of these hydrophobic waxes, plant waxes, animal waxes and synthetic and/or mineral waxes. The waxy matrix may comprise at least one oil and at least one hydrophobic compound for adjusting the melting point, the hardness, the physicochemical properties and the biological properties such as the biodegradability. The particles may also contain soluble or insoluble additives or active agents such as mineral particles. According to the invention, mixtures with a melting point of between 20° C. and 65° C., preferentially between 32° C. and 48° C., are used as waxy matrix.

Use may be made of triglycerides of C8 to C30 fatty acids, fractionated triglycerides, modified triglycerides, synthetic triglycerides, triglyceride mixtures, medium-chain and long-chain triglycerides and structured triglycerides. It is also possible to use other waxes such as fatty alcohols of high molecular weight, fatty acids with a hydrocarbon-based chain which is preferentially linear and saturated, fatty acids with an even number of carbon atoms of from C12 to C30, esters of acids and of alcohols of high molecular weight, notably esters of C8 to C30 fatty acids and of C2 to C32 alcohols. In all cases, the mixture obtained is solid, notably at room temperature, and characterized by the absence of surfactant compounds, by hydrophobic behaviour, non-wettability with water and the absence of hygroscopicity. The fatty acids that may be used in the present invention are non-neutralized and non-ionized fatty acids (i.e. they are exclusively in carboxylic acid form —COOH).

Advantageously, the waxy matrix comprises, and notably consists of, at least one fatty substance chosen from the group formed from plant waxes, notably from carnauba wax, candelilla wax, esparto grass wax, olive wax, rice wax, jojoba wax, hydrogenated jojoba wax and absolute waxes of flowers, beeswaxes, modified beeswaxes, paraffin and paraffin derivatives, ozokerite, polyolefins, non-neutralized and non-ionized fatty acids (i.e. exclusively in carboxylic acid form —COOH), fatty acid esters -notably chosen from the group formed from esters of linear-chain fatty acids with a number of carbon atoms of between 4 and 30, notably lauric acid esters, myristic acid esters, palmitic acid esters and stearic acid esters- triglycerides, triglyceride derivatives, palm oil and cocoa butter.

The waxy matrix comprises, and notably consists exclusively of, at least one wax chosen from the group formed from the following triglycerides and derivatives, without the list being limiting: tribehenate, glyceryl tributyrate, glyceryl tricaproate, glyceryl tricaprylate, glyceryl tricaprate, glyceryl triundecanoate, glyceryl trilaurate, glyceryl trimyristate, glyceryl tripalmitate, glyceryl tristearate, glyceryl trimyristoleate, glyceryl tripalmitoleate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, glyceryl tricaprylate/laurate/stearate, glyceryl 1,2-caprylate-3-linoleate, glyceryl 1,2-caprate-3-stearate, glyceryl 1,2-laurate-3-myristate, glyceryl 1,2-myristate-3-laurate, glyceryl 1,3-palmitate-2-butyrate, glyceryl 1,3-stearate-2-caprate, glyceryl 1,2-linoleate-3-caprylate. Mention may also be made of palm oil, carnauba wax, candelilla wax, esparto grass wax, cocoa butter, ozokerite, plant waxes such as olive wax, rice wax, hydrogenated jojoba wax or absolute waxes of flowers. Beeswaxes and modified beeswaxes may also be used as waxes of natural origin. The fatty acids that may be used according to the invention are in acid form (i.e. in carboxylic acid form —COOH), for instance myristic acid, lauric acid, palmitic acid or stearic acid. Since the salts of these fatty acids form soaps, they cannot in any way be used since they would promote the wettability of the waxy matrix.

To improve the solubility or the dispersion of said at least one digestive enzyme in the matrix, it is occasionally necessary to add an oil. This makes it possible to adjust the melting point. Mention may also be made of oily triglycerides such as plant oils, certain fish oils, hydrogenated plant oils and partially hydrogenated plant oils.

The pharmaceutical commercial compounds denoted as “hard fat”, consisting essentially of triglycerides, may also be used. Since residual traces of monoglycerides and diglycerides may be present therein, it should then be checked that the strictly hydrophobic and non-wettable nature is indeed respected in order to be able to use these products as starting material for the matrix. Such is the case for the products named Suppocire® AM, CM, DM and D.

Besides the waxes mentioned above, the waxy matrix of the solid lipid particles according to the invention may contain an oil or a mixture, for example a hydrophobic silicone oil, squalene, a derivative thereof and esters thereof.

Other oily compounds such as oleyl alcohol, sunflower oil, palm oil, olive oil, non-neutralized and non-ionized fatty acids (i.e. in carboxylic acid form —COOH) and fatty alcohols may be used, but the mixture obtained must be characterized by hydrophobic behaviour, absence of miscibility with water. A person skilled in the art knows that, for the components of the waxy matrix, the melting point by heating of the waxy matrix must not exceed the degradation temperature of the various compounds.

Other compounds may be added to the waxy matrix, such as paraffins. Mention may be made of fillers, such as talc, kaolin or mica, colorants, and agents for adjusting the appearance, the colour, the density and the hardness of the waxy matrix. The waxy matrix contains a digestive enzyme or a mixture of digestive enzymes which may be in a dispersed form or in a dissolved form in the waxy matrix or may be in both forms.

According to a particular embodiment of the invention, the waxy matrix may be formed from a mixture of waxes.

Advantageously and according to the invention, the formulation contains fatty acids (non-neutralized and non-ionized, i.e. in carboxylic acid form -COOH) or fatty acid esters in a mass proportion of between 0.5% and 75% of the formulation, preferentially between 1% and 30% of the formulation. Throughout the text, the term “fatty acid(s)” means one or more non-neutralized and non-ionized fatty acids (i.e. in carboxylic acid form —COOH).

Advantageously and according to the invention, each solid lipid particle comprises only one digestive enzyme.

Advantageously and according to the invention, the formulation is formed from a mixture of solid lipid particles, each solid lipid particle comprising only one digestive enzyme, the mixture of solid lipid particles forming a mixture of digestive enzymes.

The invention also relates to a galenical preparation comprising a formulation according to the invention.

Advantageously, the galenical preparation comprises a formulation according to the invention and at least one additional compound chosen from the group formed from lubricants, notably talc, homogenizing agents, notably silica, pharmaceutically acceptable binders, stabilizers, breakdown agents, colorants, preserving agents, sweeteners, and thickeners, notably at least one cellulose derivative.

Advantageously, said additional compound is in a mass proportion of between about 0.1% and 99% in the galenical composition, preferentially between 3% and 32%.

Advantageously, the galenical preparation is in a form chosen from the group formed from a powder, notably a powder for reconstituting a dispersion by addition of water, said powder being contained in a unit or multiple container in the form of a sachet, or of a glass or polymeric or metallic bottle, a tablet, a lozenge, notably a lozenge obtained by freeze-drying, a gel capsule or a wafer capsule, notably a tablet, a lozenge, a gel capsule or a wafer capsule packaged in a blister pack or in a packaging bag.

The term “mixture containing the active compound” or “matrix containing the active agent” or “product containing the active agent” denotes the result of mixing of the constituents of the waxy matrix, of hydrophobic excipients and of said at least one digestive enzyme after melting.

The invention also relates to a process for preparing a formulation according to the invention.

In a preferential embodiment, the formulation is prepared according to a process comprising the following steps:
a/melting of the waxy matrix with stirring, followed by reducing the temperature of the waxy matrix to a temperature at least 3° C. above the melting point of the waxy matrix,
b/addition and dispersion of said at least one digestive enzyme in the molten waxy matrix,
c/solidification of the mixture formed by cooling to a temperature at least 15° C. below the melting point of the waxy matrix,
e/ mechanical milling at a temperature at least 10° C. and preferentially 20° C. below the melting point of the solidified mixture (of the waxy matrix containing said at least one digestive enzyme), whereby the formulation of solid lipid particles in powder form is formed.

Advantageously, the process according to the invention comprises a step d/of premilling the cooled mixture formed on conclusion of step c/.

In a process according to the invention, during step a/, the hydrophobic matrix is prepared by melting the excipients followed by stirring and reducing the temperature to at least 3° C. and preferentially 5° C. above the melting point of the final mixture.

During step b/, the digestive enzyme is added to and dispersed in the molten matrix.

During step c/, the matrix is recovered and is solidified by cooling to at least 15° C. below the melting point of the mixture obtained.

During step d/, premilling is performed.

During step e/, milling is performed at a temperature at least 10° C. and preferentially 20° C. below the melting point of the matrix.

During step f/, the milled pancrelipase powder is recovered.

In a step b/of a process according to the invention, said at least one digestive enzyme is dispersed as active principle in a wax or a mixture of waxes and of strictly hydrophobic excipients, named hydrophobic waxy matrix, which has been melted beforehand Said at least one digestive enzyme is protected against any contact with oxygen and water and more generally against any external chemical stress. Said at least one digestive enzyme is added to the waxy matrix at a temperature of the waxy matrix that is notably at least 3° C., preferably 5° C., above the melting point of the waxy matrix, but still below the degradation temperature or deactivation temperature of said at least one digestive enzyme. The liquid waxy matrix obtained, containing said at least one digestive enzyme, is then solidified by cooling and then premilled.

The solidified mixture is then milled in order to obtain a ready-to-use powder of controlled particle size. This powder of solid lipid particles may be dispersed in water without any risk to the digestive enzyme(s) on account of its strictly hydrophobic nature.

This mode of preparation of the formulation is thus rapid and does not require any chemical modifications of the digestive enzyme(s) or any surface treatment of the solid lipid particles or of the crystals of the digestive enzyme(s). It enables the digestive enzyme(s) to be incorporated into the formulation from the very first phase of mixing of the various constituents of the formulation. It is inexpensive and easy to perform. The implementation of the process for preparing a formulation according to the present invention does not involve any addition of emulsifiers or of amphiphilic products, nor does it require any organic solvents, the removal of which always remains difficult and the use of which is increasingly restrictive. It does not involve any shear-thinning agent either. It does not involve any contact with water or with any aqueous composition during the process, avoiding any dissolution of water-soluble digestive enzyme(s) in this aqueous composition and any extraction of water-soluble digestive enzyme(s). The digestive enzyme(s) are uniformly distributed in the waxy matrix of the solid lipid particles, even at the surface of said particles, without a concentration gradient, notably without a radial concentration gradient.

The mixing of the various components of the waxy matrix and of said at least one digestive enzyme is performed in a thermostatically regulated reactor or a melting vessel. The component of the waxy matrix which has the highest melting point is melted first, with mechanical stirring suitable for dispersion of all the components.

This process is thus rapid and does not require a long and difficult stirring step.

The mixture is then immediately cooled so as to protect the sensitive digestive enzyme(s) and to obtain a solid phase formed from the waxy matrix and the digestive enzyme(s). According to a particular embodiment of the invention, for amounts of less than 1 kg, the cooling of the molten waxy matrix containing the dispersed digestive enzyme(s) may be performed by spreading on contact cooling plates. Solidification is obtained in less than 120 seconds, and the plates are then placed in a cold chamber, which enables the steps to be uncoupled, if necessary.

For larger amounts, cooling may be performed by passing through a continuous cooling system. The molten product is deposited in the form of droplets, filaments or a film onto a stainless-steel belt conveyor which passes through a cooling chamber and is recovered in solid form at the outlet of the cooling chamber. Other systems such as those equipped with a refrigerated cylinder may be used.

The cooling temperature of the matrix is controlled to at least 15° C. below the melting point of the mixture. According to one embodiment of the invention, the cooling temperature is between −195° C. and 30° C. and preferentially between -10° C. and 5° C.

The waxy matrix is then cooled by contact or convection before being poured into the premilling system in order to obtain fragments preferentially less than 40 mm in size, in order to correctly feed the mill. Premills or any other suitable system may be used for this purpose. Certain solidified mixtures are fragile and can be premilled and milled in the same machine in a single step, but at different speeds.

Advantageously and according to the invention, at least one from among the solidification step c), the premilling step d) and the milling step e) is performed by cooling with cardice or liquid nitrogen.

In a final step according to the invention, the premilled matrix is reduced to powder by milling A large number of mills may be used, such as hammer, knife, rotor, ball or jet mills. To facilitate the milling of the waxy matrix, one embodiment consists in cooling the matrix to harden and embrittle it. This operation takes place before or during the milling step.

The powder of solid lipid particles comprising the waxy matrix obtained on conclusion of the milling can be packaged directly. This powder according to the invention has a particle size of between 50 and 1200 microns (μm) and preferentially between 150 and 800 microns (μm). In a preferential embodiment, the powder has a particle size of between 250 and 550 microns (μm).

The preparation of the formulation according to the invention does not involve a step of dispersing a waxy matrix in a liquid or gelled aqueous phase. The formulation according to the invention and the solid lipid particles are free of water at the surface and deep down, and of any water-soluble polymer residue, in particular of water-soluble surface polymer(s), in particular shear-thinning polymers, which are liable to interact with said at least one digestive enzyme and to affect the physicochemical stability thereof, and to reduce the storage time and the degree of filling

The invention also covers a galenical preparation comprising a formulation according to the invention. Advantageously and according to the invention, the galenical preparation contains at least one additional compound chosen from the group formed from lubricants, homogenizing agents, pharmaceutically acceptable binders, stabilizers, breakdown agents, colorants, preserving agents, sweeteners and thickeners.

Advantageously and according to the invention, said additional compound is in a mass proportion of between about 0.1% and 99% in the galenical composition. The filling capacity of the waxy matrix may extend from 0.1% to 90% relative to the weight. A person skilled in the art knows that when these components are incorporated into the waxy matrix according to the invention, a suitable hydrophobic waxy composition should be chosen so that the process can be performed.

The galenical preparation may be in a conventional galenical form such as a powder, a gel capsule, a wafer capsule, a tablet, a tablet, notably an orodispersible tablet, a lozenge, notably a lozenge obtained by freeze-drying, or an aqueous suspension. It may be presented in the form of a dispersible powder packaged in a sachet or a bottle for obtaining a liquid form.

Advantageously, the galenical preparation contains at least one additional compound or additive chosen from the group formed from lubricants, for example talc, homogeneity-enhancing agents, for instance silica, pharmaceutically acceptable binders, stabilizers, breakdown agents, disintegrants, colorants, preserving agents, sweeteners, diluents, and thickeners, for instance cellulose derivatives.

Examples of suitable binders include starches, sugars such as lactose, sugar alcohols, xylitol, sorbitol, maltitol, cellulose, modified celluloses, hydroxypropylcellulose (HPC), sodium carboxymethylcellulose (CMC), alginic acid, polyvinylpyrrolidone (PVP), and mixtures thereof.

Examples of suitable disintegrants that may be mentioned include dibasic calcium phosphate, alginic acid, HPC, CMC, swellable ion-exchange resins, alginates, formaldehyde-casein, cellulose, croscarmellose sodium, crospovidone, cellulose, sodium carboxymethylstarch, starch glycolate, and mixtures thereof. Examples of suitable lubricants include calcium stearate, magnesium stearate, sodium stearyl fumarate, stearic acid, zinc stearate, talc, waxes, Sterotex® and mixtures thereof; examples of homogeneity-promoting agents include powders such as colloidal silicon dioxide, talc, and mixtures thereof.

By way of example, diluents that may be mentioned include microcrystalline cellulose, for example Pharmacel 112 from DFE Pharma, starch, calcium phosphate, lactose, such as Pharmatol, sucrose, magnesium carbonate, mannitol, sorbitol, and combinations thereof. In another embodiment, the oral compositions or the oral dosage forms of the present invention may comprise a combination of diluents such as microcrystalline cellulose, starch and lactose.

The mass proportion of diluent may be between about 0.1% and 99% and preferentially between 3% and 32% of the galenical preparation.

In another embodiment, the oral compositions or oral dosage forms of the present invention may comprise a combination of breakdown agents such as microcrystalline cellulose and sodium starch glycolate or croscarmellose sodium and crospovidone.

The mass proportion of disintegrant may be between about 0.1% and 25% and preferentially between 0.5% and 6% of the galenical preparation.

The powder obtained according to the invention may be packaged in unit or multiple forms, for example in the form of a bag, a bottle, a sachet or a blister pack.

The invention also relates to a formulation, to a process for preparing such a formulation and to a galenical preparation, characterized, in combination or separately, by all or some of the characteristics mentioned hereinabove or hereinbelow. Irrespective of the formal presentation that is given thereof, unless explicitly indicated otherwise, the various characteristics mentioned hereinabove or hereinbelow should not be considered as being strictly or inextricably linked together, the invention being able to concern only one of these structural or functional characteristics, or only a portion of these structural or functional characteristics, or only a portion of one of these structural or functional characteristics, or else any group, combination or juxtaposition of all or a portion of these structural or functional characteristics.

Other aims, characteristics and advantages of the invention will emerge on reading the following description of some of the possible embodiments thereof, and the examples, which are given without any implied limitation.

The examples that follow illustrate the invention. For some of the following examples, the taste- and odour-masking tests were performed on a sample of 10 individuals. The test products are never absorbed.

The results are expressed according to the following scale:
1: the taste of the active principle is not detected,
2: the taste of the active principle is sparingly perceived,
3: the taste of the active principle is detected,
4: the taste of the active principle is still acceptable,
5: the taste of the active principle is unacceptable.

The test value is calculated by taking the mean of the grades obtained out of a maximum total of 10 (/10).

The lipase activity is determined according to the method of the International Pharmaceutical Federation/European Pharmacopoeia (abbreviated hereinbelow as FIP/Ph.Eur.). In this standard analytical method, the hydrolytic activity of the lipase in the sample to be studied is determined using olive oil (USP) as substrate. The free fatty acids released from the olive oil triglycerides are titrated with a sodium hydroxide solution at a constant pH of 9.0. The lipase activity of the sample is determined by comparing the rate at which the sample hydrolyses an olive oil emulsion with the rate at which a suspension of a pancreas powder reference standard hydrolyses the same substrate under the same conditions.

### EXAMPLE 1

Preparation of a stabilized, taste-masked, gastro-protected, rapid-release pancreatin formulation.

Composition:

| | | |
|---|---|---|
| PPM1 triglycerides (Stéarinerie Dubois) | 0.94 | kg |
| wax | 0.1 | kg |
| pancreatin | 1.625 | kg |
| talc | 0.020 | kg |
| silica | 0.005 | kg |

The compound with the highest melting point is brought 5° C. above its melting point in a 5 litre thermostatically regulated reactor, and the various compounds are then gradually added from the highest melting point to the lowest. The temperature of the mixture is gradually lowered and then maintained 3° C. above the melting point of the new mixture obtained, 48° C. in this instance. The pancreatin is added last. Dispersion of the pancreatin in the molten waxy matrix is performed using a stirring system equipped with an anchor-shaped spindle, at a speed of 200 rpm. Stirring is then performed using a T25 Turrax turbomixer at 4500 rpm for 6 minutes to obtain complete dispersion.

The waxy matrix containing the pancreatin is solidified by pouring onto a rotating cylinder equipped with a doctor blade, the temperature of which is 0° C.

The recovered fragments, with a mean size of 4 mm, are then cooled with cardice. The mixture is premilled and then milled using a stainless-steel Retsch GM knife mill
Speed: 3000 (rpm),
Time: 90 seconds
Particle size: mean diameter of 565 microns (μm).

This powder comprising pancreatin is then evaluated in a taste test. The result is a mean value of 1.20 in the taste test.

Since the mean value is less than 2, the pancreatin taste and odour are not detectable and no sensation of mucosal irritation is observed.

### EXAMPLE 2

Powder preparation containing gastro-protected, stabilized, taste-masked pancreatin with accelerated intestinal release. This formulation is intended for the production of a product intended for treating exocrine pancreatic insufficiency.

Composition:

| | | |
|---|---|---|
| PPM1 triglycerides (Stéarinerie Dubois) | 0.69 | kg |
| stearic acid | 0.058 | kg |
| pancreatin | 1.325 | kg |
| talc | 0.020 | kg |
| silica | 0.005 | kg |
| mint flavouring | 0.009 | kg |

The powder is prepared according to the protocol described in Example 1. The powder obtained is characterized by the absence of detection of the pancreatin taste, with a score of 1.2 in the taste test.

The powder obtained is characterized by a pronounced mint taste, with a score of 5 in the taste test.

### EXAMPLE 3

Preparation of an oral-route hydro-dispersible powder containing particles filled with digestive enzymes.

Composition:

| | | |
|---|---|---|
| particles according to Example 1 | 100 | g |
| red berry flavouring | 8 | g |
| aspartame | 4 | g |
| xanthan gum (Xanthural 180) | 1 | g |
| talc | 0.5 | g |
| colloidal silica | 0.1 | g |

The constituents of the composition are placed in a Turbula powder mixer (WAB France). After mixing, the powder is divided into 0.25 g unit sachets. Uptake in 50 ml of water allows an aqueous dispersion to be reconstituted. The taste test performed on the dispersion gives no detection of the active principle.

### EXAMPLE 4

Preparation of particles containing pancrelipase

Composition:

| | | |
|---|---|---|
| mixture of triglycerides (m.p. 45° C.) | 700 | g |
| DUB PP triglycerides (Stéarinerie Dubois) | 100 | g |
| pancreatin | 1600 | g |
| talc | 10 | g |
| sodium hydrogen carbonate | 5 | g |
| silica (Aerosil) | 2 | g |

The compound with the highest melting point, the DUB PP triglycerides, is brought to 50° C. in a 500 ml thermostatically regulated reactor, and the various compounds are then gradually added from the highest melting point to the lowest. The temperature of the mixture is gradually lowered and then maintained at 45° C. During the addition of the composition, the stirring speed of the three-blade impeller is 100 rpm.

The pancreatin is added last. Dispersion of the pancreatin in the lipid phase is performed using a T25 Turrax brand turbomixer stirring system at a speed of 6000 rpm. The matrix is solidified by pouring onto a stainless-steel rotating drum 55 cm in diameter, the temperature of which is maintained at -10° C., rotating at 5 rpm and equipped with a tangential blade for detaching the matrix solidified on the surface. The recovered fragments, with a mean size of 25 mm, are premilled and then milled using a Retsch GM200 knife mill co-filled with cardice, under the following conditions:
premilling: 20 seconds at 1500 rpm
milling: 70 seconds at 3000 rpm

The particles thus obtained have a mean size of 482 microns (pm).

Stability test in 0.1N hydrochloric acid solution: The stability of the formulation is determined by monitoring the change in activity of the lipase as a function of time. The results are expressed as a percentage of initial activity as a function of time and are collated in table 1 below. The lipase activity is determined after neutralization to pH 7 by adding 0.02N NaOH solution.

TABLE 1

| | Incubation time 0.1N HCl | | | | | |
|---|---|---|---|---|---|---|
| | | T 5 | T 10 | T 20 | T 30 | T 60 |
| | T0 | min | min | min | min | min |
| Pancreatin | 100% | 60% | 5% | 0% | 0% | 0% |
| Formulation | 100% | 100% | 101% | 96% | 97% | 95% |
| Example 4 | | | | | | |

The pancreatin formulated according to the invention conserves at least 95% of its lipase activity after one hour of incubation at 37° C. in 0.1N hydrochloric acid medium with stirring (30 rpm).

Test of rate of release of the lipases under intestinal pH conditions. The lipase activity is measured by hydrolysis of the fatty acids released from olive oil triglycerides by titrating with a solution (0.02N NaOH) at a constant pH of 9.0 and at 37° C. The substrate is a pharmacopoeia olive oil solution, prepared according to the “FIP lipase” protocol adapted for the preparation of 400 ml:
Substrate 240 ml, of which 24.5% of olive oil emulsified with acacia gum;
H2Od: 140 ml
Sodium taurocholate solution (0.5%): 20 ml
amount of pancreatin: 500 IU.

The results are collated in table 2 below. The results are expressed in millimoles (mmol) of fa acids released.

TABLE 2

| Time (min) | 0 | 2 | 5 | 10 | 20 | 40 | 60 | 120 |
|---|---|---|---|---|---|---|---|---|
| Pancreatin | 0 | 0 | 1 | 4 | 11 | 37 | 51 | 52 |
| | | | mmol | mmol | mmol | mmol | mmol | mmol |
| Formulation | 0 | 0 | 1 | 3 | 9 | 31 | 42 | 44 |
| Example 4 | | | mmol | mmol | mmol | mmol | mmol | mmol |
| Creon 6000 | 0 | 0 | 0 | 1 | 2 | 7 | 18 | 36 |
| | | | | mmol | mmol | mmol | mmol | mmol |

At 20 minutes, the amount of fatty acid released is four times greater with formulation 5 according to the invention than with the commercial pancreatin formulation based on the coating named “enteric coating”.

The invention may be the subject of numerous variants and applications other than those described hereinabove. In particular, it goes without saying that, unless otherwise indicated, the various structural and functional characteristics of each of the embodiments described hereinabove must not be considered as combined and/or strictly and/or inextricably linked to each other, but, on the contrary, as simple juxtapositions. In addition, the structural and/or functional characteristics of the various embodiments described hereinabove may form the subject totally or partly of any different juxtaposition or of any different combination.

1/ The formulation of at least one digestive enzyme, the formulation comprising:
solid lipid particles, the solid lipid particles being:
between 50 μm and 1200 μm in size,
strictly hydrophobic,
free of water, of organic solvent, of surfactant compound and of polymer, and comprising:
at least one digestive enzyme,
a strictly hydrophobic, non-hygroscopic waxy matrix, in which said at least one digestive enzyme:
has homogeneous distribution in each solid lipid particle of the formulation,
is distributed without a distribution gradient towards the interior of the waxy matrix, and
represents between 0.1% and 90% of the mass of the formulation, the formulation having a melting point of between 20° C. and 65° C.

2/ The formulation according to claim 1, wherein the solid lipid particles have a size of between 150 μm and 800 μm and notably between 250 μm and 550 μm.

3/ The formulation according to claim 1, wherein the melting point is between 20° C. and 55° C.

4/ The formulation according to claim 1, wherein the waxy matrix consists of at least one fatty substance chosen from the group formed from plant waxes, beeswaxes, modified beeswaxes, paraffin and paraffin derivatives, ozokerite, polyolefins, fatty acids, fatty acid esters, triglycerides, triglyceride derivatives, palm oil and cocoa butter.

5/ The formulation according to claim 1, wherein the formulation contains fatty acids or fatty acid esters in a mass proportion of between 0.5% and 75% of the formulation.

6/ The formulation according to any one of claim 1, wherein each solid lipid particle comprises only one digestive enzyme.

7/ The formulation according to claim 1, wherein the formulation is in the form of a mixture of solid lipid particles, each solid lipid particle comprising only one digestive enzyme, the mixture of solid lipid particles forming a mixture of digestive enzymes.

8/ A galenical preparation comprising a formulation according to claim 1, wherein said galenical contains at least one additional compound chosen from the group formed from lubricants, homogenizing agents, pharmaceutically acceptable binders, stabilizers, breakdown agents, disintegrants, colorants, preserving agents, sweeteners, diluents and thickeners.

9/ The galenical preparation according to claim 8, wherein said additional compound is in a mass proportion of between about 0.1% and 99% in the galenical composition.

10/ The galenical preparation according to claim 8, wherein said galenical is in a form chosen from the group formed from a powder, a tablet, a lozenge, a gel capsule and a wafer capsule.

11/ A process for preparing a formulation according to claim 1, wherein said process comprises the following steps:
a/ melting of the waxy matrix with stirring, followed by reducing the temperature of the waxy matrix to a temperature at least 3° C. above the melting point of the waxy matrix,
b/ addition and dispersion of said at least one digestive enzyme in the molten waxy matrix,
c/ solidification of the mixture formed by cooling to a temperature at least 15° C. below the melting point of the waxy matrix,
e/ mechanical milling at a temperature at least 10° C. and preferentially 20° C. below the melting point of the solidified mixture, whereby the formulation of solid lipid particles in powder form is formed.

12/ The process according to claim 11, it wherein said process comprises a premilling step d/ and in that at least one step from among the solidification step c/, the premilling step d/ and the milling step e/ is performed by cooling with cardice or with liquid nitrogen.
